(19) United States
(12) Patent Application Publication
(10) Pub. No.: US 2024/0408098 A1
(43) Pub. Date: Dec. 12, 2024

(54) COMBINATIONS OF KRAS G12D INHIBITORS WITH PI3Ka INHIBITORS AND RELATED METHODS OF TREATMENT

(71) Applicant: MIRATI THERAPEUTICS, INC., San Diego, CA (US)

(72) Inventors: Jill Hallin, San Diego, CA (US); James Gail Christensen, San Diego, CA (US); Vickie Bowcut, San Diego, CA (US); Peter Olson, San Diego, CA (US)

(21) Appl. No.: 18/694,848

(22) PCT Filed: Oct. 4, 2022
(86) PCT No.: PCT/US2022/045619

§ 371 Date: Mar. 22, 2024

Related U.S. Application Data
(60) Provisional application No. 63/252,384, filed on Oct. 5, 2021.

(51) Int. Cl. A61K 31/519; A61K 31/4439; A61P 35/00

(52) U.S. Cl. CPC A61K 31/519; A61K 31/4439; A61P 35/00

(57) ABSTRACT

The present invention relates to combination therapies for treating KRas G12D cancers: in particular, the present invention relates to methods of treating cancer in a subject in need thereof, comprising administering to the subject a therapeutically effective amount of a combination of a PI3Ka inhibitor and a KRas G12D inhibitor, pharmaceutical compositions comprising a such compositions, kits comprising such compositions and methods of use thereof.

### FIELD OF THE INVENTION

The present invention relates to combination therapies useful for treating cancer. In particular, the present invention relates to therapeutically effective combinations of a KRas G12D inhibitor and a PI3Ka inhibitor, and additionally pharmaceutical compositions comprising theses agents, kits comprising such compositions, and methods of use thereof.

### BACKGROUND OF THE INVENTION

### KRas Inhibitors

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79(11):3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 33% of these KRas driver mutations in lung adenocarcinoma, with a G12D mutation being a common activating mutation (e.g., see Li, Balmain and Counter, (2018) Nat Rev Cancer Dec; 18(12):767-777; Sanchez-Vega, et al, (2018) Cell; 173, 321-337).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, only a single KRas G12C inhibitor (the KRas G12C inhibitor sotorasib) has demonstrated sufficient safety and/or efficacy to obtain regulatory approval (e.g., see: FDA Approves First KRAS Inhibitor: Sotorasib. [No authors listed] Cancer Discov. 2021 August; 11(8):OF4. doi: 10.1158/2159-8290.CD-NB2021-0362. Epub 2021 Jun. 22). To date, no KRas G12D inhibitors have demonstrated sufficient safety and/or efficacy to obtain regulatory approval.

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well as those that target KRas G12D (e.g., see K-Ras(G12D) Has a Potential Allosteric Small Molecule Binding Site, Feng H, Zhang Y, Bos P H, Chambers J M, Dupont M M, Stockwell B R, Biochemistry, 2019 May 28; 58(21):2542-2554. doi: 10.1021/acs.biochem.8b01300. Epub 2019 May 14; and Second harmonic generation detection of Ras conformational changes and discovery of a small molecule binder, Donohue E, Khorsand S, Mercado G, Varney K M, Wilder P T, Yu W, MacKerell A D Jr, Alexander P, Van Q N, Moree B, Stephen A G, Weber D J, Salafsky J, McCormick F., Proc Natl Acad Sci USA 2019 Aug. 27; 116(35):17290-17297, doi: 10.1073/pnas.1905516116. Epub 2019 Aug. 9). Clearly there remains a continued interest and effort to develop inhibitors of KRas, particularly inhibitors of activating KRas mutants, including KRas G12D.

While the KRas G12D inhibitors disclosed herein are potent inhibitors of KRas G12D signaling and exhibit single agent activity inhibiting the in vitro proliferation of cell lines harboring a KRas G12D mutation, the relative potency and/or observed maximal effect of any given KRas G12D inhibitor can vary between KRas mutant cell lines. The reason or reasons for the range of potencies and observed maximal effect is not fully understood but certain cell lines appear to possess differing intrinsic resistance. Thus, there is a need to develop alternative approaches to maximize the potency, efficacy, therapeutic index and/or clinical benefit of KRas G12D inhibitors in vitro and in vivo.

### PI3Ka Inhibitors

Phosphoinositide 3-kinase inhibitors (PI3K inhibitors) are a class of medical drug that functions by inhibiting one or more of the phosphoinositide 3-kinase enzymes, which are part of the PI3K/AKT/mTOR pathway, an important signalling pathway for many cellular functions such as growth control, metabolism and translation initiation. Within this pathway there are many components, inhibition of which may result in tumor suppression.

There are a number of different classes and isoforms of PI3Ks.Class 1 PI3Ks have a catalytic subunit known as p110, with four types (isoforms), one of which is p110 alpha, or PI3Ka or PI3KA. PI3K inhibitors, including PI3Ka inhibitors, are being actively investigated for treatment of various cancers, and also inflammatory respiratory disease.

The phosphatidylinositol-4,5-bisphosphate 3-kinase, catalytic subunit alpha (the HUGO-approved official symbol=PIK3CA; HGNC ID, HGNC:8975), also called p110α protein, is a class I PI 3-kinase catalytic subunit. The human p110α protein is encoded by the PIK3CA gene.

Phosphatidylinositol-4,5-bisphosphate 3-kinase (also called phosphatidylinositol 3-kinase (PI3K)) is composed of an 85 kDa regulatory subunit and a 110 kDa catalytic subunit. The protein encoded by this gene represents the catalytic subunit, which uses ATP to phosphorylate phosphatidylinositols (PtdIns), PtdIns4P and PtdIns(4,5)P2. The involvement of p110α in human cancer has been hypothesized since 1995. Support for this hypothesis came from genetic and functional studies, including the discovery of common activating PIK3CA missense mutations in common human tumors. It has been found to be oncogenic and is implicated in cervical cancers. PIK3CA mutations are present in over one-third of breast cancers, with enrichment in the luminal and in human epidermal growth factor receptor 2-positive subtypes (HER2+). The three hotspot mutation positions (GLU542, GLU545, and HIS1047) have been widely reported. While substantial preclinical data show an association with robust activation of the pathway and resistance to common therapies, clinical data do not indicate that such mutations are associated with high levels of pathway activation or with a poor prognosis. It is unknown whether the mutation predicts increased sensitivity to agents targeting the P3K pathway.

Pharmaceutical companies are designing and characterizing potential p110α isoform specific inhibitors. One such compound is BYL719, also known as Alpelisib, an oral medication approved for the treatment of certain types of breast cancer ((HR)-positive, (HER2)-negative, PIK3CA-mutated, advanced or metastatic) with the chemotherapeutic fulvestrant.

While PI3Ka inhibitors such as BYL719 and others are potent anti-cancer agents that exhibits activity alone and with chemotherapeutic agents, the relative potency and/or observed maximal effect of PI3Ka-based regimens can vary. The reason or reasons for such variation is not fully understood but certain cell lines appear to possess differing intrinsic resistance. Thus, there is a need to develop alternative approaches to maximize the potency, efficacy, therapeutic index and/or clinical benefit of PI3Ka inhibitors.

### SUMMARY OF THE INVENTION

The combination therapy of the present invention, in one aspect, increases the potency of KRas G12D inhibitors resulting in improved efficacy of KRas G12D inhibitors disclosed herein. The combination therapy of the present invention, in another aspect, provides improved clinical benefit to patients compared to treatment with KRas G12D inhibitors disclosed herein as a single agent.

Thus in one aspect of the invention there are provided therapeutically effective combinations of a PI3Ka inhibitor or a pharmaceutically acceptable salt thereof, for instance the PI3Ka inhibitors recited in U.S. Pat. Nos. 8,227,462 and 8,476,268, including but not limited to BYL719 (alpelisib):

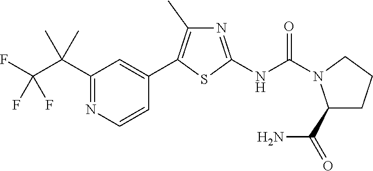 [SMILES: Cc1nc(NC(=O)N2CCC[C@H]2C(N)=O)sc1-c1ccnc(C(C)(C)C(F)(F)F)c1]

(2S)-N1-[4-Methyl-5-[2-(2,2,2-trifluoro-1,1-dimethylethyl)-4-pyridinyl]-2-thiazolyl]-1,2-pyrrolidinedicarboxamide
   or a pharmaceutically acceptable salt thereof, and the KRas G12D inhibitor compound MRTX1133:

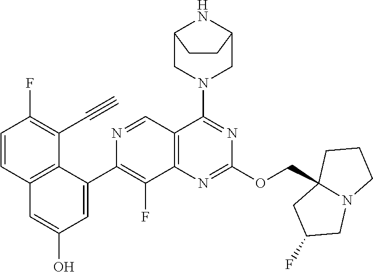 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

4-(4-((1R,5S)-3,8-diazabicyclo[3.2.1]octan-3-yl)-8-fluoro-2-(((2R,7aS)-2-fluorohexahydro-1Hpyrrolizin-7a-yl)methoxy)pyrido[4,3-d]pyrimidin-7-yl)-5-ethynyl-6-fluoronaphthalen-2-ol
   or a pharmaceutically acceptable salt thereof.

In another aspect of the invention there are provided therapeutically effective combinations of a PI3Ka inhibitor or a pharmaceutically acceptable salt thereof, for instance BYL719, inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine), or a pharmaceutically acceptable salt thereof; and the KRas G12D inhibitor MRTX1133 or MRTX1133 analogs, or a pharmaceutically acceptable salt thereof for instance the compounds disclosed and described in WIPO publication WO2021/041671, including but not limited to: Ex. 252 (MRTX1133), 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynyl-6-fluoronaphthalen-2-ol; Ex. 243, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; Ex. 246, 4-(4-((1R,5S)-3,8-diazabicyclo[3.2.1]octan-3-yl)-8-fluoro-2-(((2R,7aS)-2-fluorohexahydro-1H-pyrrolizin-/a-yl)methoxy)pyrido[4,3-d]pyrimidin-7-yl)-5,6-difluoronaphthalen-2-ol; Ex. 251, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; Ex. 253, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; Ex. 259, 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and Ex. 282, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; or a pharmaceutically acceptable salt thereof and a pharmaceutically acceptable excipient.

Additional PI3Ka inhibitors or a pharmaceutically acceptable salt thereof include: alpelisib, GDC-0077, YM-201636, serabelisib, PIK-75 hydrochloride, GDC-0326, HS-173, A66, PF-4989216, pilaralisib analogue, PI-828, brevianamide F, PI3Ka-IN-4, BEBT-908, WYE-687, PF-06843195, CYI133, PI3Kf inhibitor 4, KU-0060648, WYE-687 dihydrochloride, PIK-75, PI3Ka/mTOR-IN-1, LY294002, AS-041164, idelalisib, buparlisib, dactolisib, pictilisib, eganelisib, copanlisib, duvelisib, fimepinostat, omipalisib, PI-103, tasclisib, PF-04691502, ZSTK474, AZD 6482, samotolisib, dactolisib tosylate, AZD8186, AS-605240, copanlisib dihydrochloride, PKI-402, apitolisib, Vps34-PIK-III, gedatolisib, PIK-93, CH5132799, bimiralisib, GSK1059615, CNX-1351, BGT226 maleate, VS-5584, sonolisib, voxtalisib, PI4KIIIbeta-IN-9, leniolisib, nemiralisib, SF2523, AZD-8835, AMG 511, AZD3458, PIK-90, pictilisib dimethanesulfonate, AS-252424, AMG319, acalisib, pilaralisib, PI-103 Hydrochloride, SAR-260301, PI-3065, PQR530, hSMG-1 inhibitor 11j, GNE-477, PI3K/mTOR Inhibitor-2, buparlisib hydrochloride, MSC2360844, SRX3207, NSC781406, TG 100713, AS-604850, IPI-3063, PF-04979064, ETP-46321, GNE-493, PIK-294, (S)-PI3Ka-IN-4, PKI-179, PIK-293, CAL-130 hydrochloride, BGT226, PI3K-IN-6, PI3K6-IN-8, FD223, PARP/PI3K-IN-1, CHMFL-PI3KD-317, PI3K/HDAC-IN-1, PI3K-IN-2, PI3K/mTOR Inhibitor-1, PKI-179 hydrochloride, hSMG-1 inhibitor 11e, NVP-BAG956, PI3Kγ inhibitor 1, ON 146040, CAL-130, BAY1082439 and AZ2.

In another aspect of the invention, pharmaceutical compositions are provided for use in the methods comprising a therapeutically effective amount of a combination of a PI3Ka inhibitor or a pharmaceutically acceptable salt thereof (for instance BYL719), and the KRas G12D inhibitor compound MRTX1133 or MRTX1133 analogs or a pharmaceutically acceptable salt thereof, for instance the compounds disclosed and described in WIPO publication WO2021/041671 including but not limited to those compounds noted above, or a pharmaceutically acceptable salt thereof and a pharmaceutically acceptable excipient.

In one aspect of the invention, provided herein are methods of treating cancer in a subject in need thereof, comprising administering to the subject a therapeutically effective amount of a combination of a PI3Ka inhibitor or a pharmaceutically acceptable salt thereof (for instance BYL719), and the KRas G12D inhibitor MRTX1133 or a pharmaceutically acceptable salt thereof.

In another aspect of the invention, provided herein are methods of treating cancer in a subject in need thereof, comprising administering to the subject a therapeutically effective amount of a combination of a PI3Ka inhibitor or a pharmaceutically acceptable salt thereof (for instance BYL719), and a KRas G12D inhibitor (for instance MRTX1133 or a MRTX1133 analog) or a pharmaceutically acceptable salt or a pharmaceutical composition thereof.

In one embodiment, the cancer is a KRas G12D-associated cancer. In one embodiment, the KRas G12D-associated cancer is pancreatic, colon, endometrial, and non-small cell lung cancer.

In some aspects of the invention, a PI3Ka inhibitor or a pharmaceutically acceptable salt thereof (for instance BYL719) and the KRas G12D inhibitor compound (such as MRTX1133) are the only active agents in the provided compositions and methods.

Besides the PI3Ka inhibitor BYL719 and the compounds noted above from U.S. Pat. Nos. 8,227,462 and 8,476,268, examples of PI3Ka inhibitors and salts suitable for the provided compositions and methods include, but are not limited to: inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine); or a pharmaceutically acceptable salt thereof.

Besides MRTX1133, examples of KRas G12D inhibitors suitable for the provided compositions and methods include, but are not limited to: 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalcn-2-ol; and pharmaceutically acceptable salts thereof.

In yet another aspect, the invention provides for methods for increasing the sensitivity of a cancer cell to a KRas G12D inhibitor, comprising contacting the cancer cell with a therapeutically effective amount of a combination of a PI3Ka inhibitor or a pharmaceutically acceptable salt thereof (for instance BYL719), and a KRas G12D inhibitor compound such as MRTX1133 (or a MRTX1133 analog) or a pharmaceutically acceptable salt thereof, wherein the PI3Ka inhibitor or salt increases the sensitivity of the cancer cell to the KRas G12D inhibitor. In one embodiment, the contacting is in vitro. In one embodiment, the contacting is in vivo.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of a PI3Ka inhibitor or a pharmaceutically acceptable salt thereof (for instance BYL719), and a KRas G12D inhibitor such as MRTX1133 or a MRTX1133 analog, or a pharmaceutically acceptable salt thereof, wherein the PI3Ka inhibitor or salt increases the sensitivity of the KRas G12D-associated cancer to MRTX1133 or a MRTX1133 analog.

Also provided herein are kits comprising a PI3Ka inhibitor or a pharmaceutically acceptable salt thereof (for instance BYL719), and the KRas G12D inhibitor compound MRTX1133 or a MRTX1133 analog, or a pharmaceutically acceptable salt or a pharmaceutical composition thereof. Also provided is a kit comprising a PI3Ka inhibitor or a pharmaceutically acceptable salt thereof (for instance BYL719), and the KRas G12D inhibitor compound MRTX1133 or a MRTX1133 analog, or a pharmaceutically acceptable salt thereof, for use in treating a KRas G12D cancer.

In a related aspect, the invention provides a kit containing a dose of a PI3Ka inhibitor or a pharmaceutically acceptable salt thereof (for instance BYL719), and the KRas G12D inhibitor compound MRTX1133 or a MRTX1133 analog or a pharmaceutically acceptable salt or a pharmaceutical composition thereof, in an amount effective to inhibit proliferation of cancer cells in a subject. The kit in some cases includes an insert with instructions for administration of the PI3Ka inhibitor or salt, and the KRas G12D inhibitor compound MRTX1133 or a MRTX1133 analog or a pharmaceutically acceptable salt or a pharmaceutical composition thereof. The insert may provide a user with one set of instructions for using the PI3Ka inhibitor or salt in combination with the KRas G12D inhibitor compound MRTX1133 or a MRTX1133 analog or a pharmaceutically acceptable salt or a pharmaceutical composition thereof.

Evidence that deeper anti-tumor responses can be achieved when inhibition of KRAS signaling is coupled with inhibition of PI3K signaling. (see Ref: Effective Use of PI3K and MEK Inhibitors to Treat Mutant K-Ras G12D and PIK3CA H1047R Murine Lung Cancers, Engelman J, Chen L, Tan X, Crosby K, et al. Nature Medicine 2008 Dec. 14 (12); 1351-1356, doi: 10.1038/nm.1890.) The combination therapy of the present invention, in one aspect, synergistically increases the potency of KRas G12D inhibitors resulting in improved efficacy of KRas G12D inhibitors disclosed herein. The combination therapy of the present invention, in another aspect, provides improved clinical benefit to patients compared to treatment with KRas G12D inhibitors disclosed herein as a single agent.

In some aspects of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 depicts the average tumor volumes in mouse xenografts for MRTX1133, alone and in combination with RYL719 (LS180 colon cancer cell line).

FIG. 2 depicts the average tumor volumes in mouse xenografts for MRTX1133, alone and in combination with RYL719 (AsPC-1 pancreatic cancer cell line).

FIG. 3 depicts the average tumor volumes in mouse xenografts for MRTX1133, alone and in combination with BYL719 (GP2D colon cancer cell line).

FIG. 4 depicts the average tumor volumes in mouse xenografts for MRTX1133, alone and in combination with BYL719 (PANC0203 pancreatic cancer cell line).

### DETAILED DESCRIPTION OF THE INVENTION

The present invention relates to combination therapies for treating KRas G12D cancers. In particular, the present invention relates to methods of treating cancer in a subject in need thereof, comprising administering to the subject a therapeutically effective amount of a PI3Ka inhibitor or a pharmaceutically acceptable salt thereof (for instance BYL719), and the KRas G12D inhibitor MRTX1133 or MRTX1133 analog, or a pharmaceutically acceptable salt thereof, pharmaceutical compositions comprising therapeutically effective amounts of the two agents, kits comprising the compositions and methods of use thereof.

Combinations of a PI3Ka inhibitor or a pharmaceutically acceptable salt thereof (for instance BYL719) with a KRas G12D inhibitor such as MRTX1133 or MRTX1133 analog, or a pharmaceutically acceptable salt thereof, increase the potency of the KRas G12D inhibitor compound against cancer cells that express KRas G12D thereby increasing the efficacy and therapeutic index of the KRas G12D inhibitor compound or pharmaceutically acceptable salts thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Gly12Asp.

As used herein, a “KRas G12D inhibitor” refers to compounds such as those represented and depected in WO2021/041671, or pharmaceutically acceptable salts thereof, as well as in other publications. These compounds are capable of negatively modulating or inhibiting all or a portion of the enzymatic activity of KRas G12D. The KRas G12D inhibitors of the present invention interact with and non-covalently bind to KRas G12D in the switch II pocket and inhibit protein-protein interactions necessary for activation of the KRAS pathway. MRTX1133 is an example of a KRas G12D inhibitor.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, a “PI3Ka inhibitor” refers to a compound that is known to inhibit or is capable of inhibiting the activity of the phosphoinositide 3-kinase enzyme's p110 alpha sub-unit.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12D mutation (e.g., as determined using a regulatory agency-approved, e.g., FDA-approved, assay or kit). In some embodiments, the subject has a tumor that is positive for a KRas G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRas G12D mutation (e.g., where the tumor is identified as such using a regulatory agency-approved, e.g., FDA-approved, kit or assay). In some embodiments, the subject is suspected of having a KRas G12D gene-associated cancer. In some embodiments, the subject has a clinical record indicating that the subject has a tumor that has a KRas G12D mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has KRas G12D mutation using a sample (e.g., a biological sample or a biopsy sample such as a paraffin-embedded biopsy sample) from a patient (e.g., a patient suspected of having a KRas G12D-associated cancer, a patient having one or more symptoms of a KRas G12D-associated cancer, and/or a patient that has an increased risk of developing a KRas G12D-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, “an effective amount” of a compound is an amount that is sufficient to negatively modulate or inhibit the activity of the desired target, or otherwise arrest or slow proliferation of the targeted cells, i.e., phosphoinositide 3-kinase enzyme's p110 alpha sub-unit, or KRas G12D. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of KRas G12D. Such amount may be administered as a single dosage or may be administered according to a regimen, whereby it is effective.

As used herein, a “therapeutically effective amount of a combination” of two compounds is an amount that together increases the activity of the combination in comparison to the therapeutically effective amount of each compound in the combination, i.e., more than merely additive. Alternatively, in vivo, the therapeutically effective amount of the combination of a PI3Ka inhibitor or a pharmaceutically acceptable salt thereof (for instance BYL719), and the KRas G12D inhibitor compound MRTX1133 or MRTX1133 analog, or a pharmaceutically acceptable salt thereof, results in an increased duration of overall survival (“OS”) in subjects relative to treatment with only the KRas G12D inhibitor. In one embodiment, the therapeutically effective amount of the combination of a PI3Ka inhibitor or a pharmaceutically acceptable salt thereof (for instance BYL719), and the KRas G12D inhibitor compound MRTX1133 or MRTX1133 analog, or a pharmaceutically acceptable salt thereof, results in an increased duration of progression-free survival (“PFS”) in subjects relative to treatment with only the KRas G12D inhibitor. In one embodiment, the therapeutically effective amount of the combination of a PI3Ka inhibitor or a pharmaceutically acceptable salt thereof (for instance BYL719), and the KRas G12D inhibitor compound MRTX1133 or MRTX1133 analog or a pharmaceutically acceptable salt thereof, results in increased tumor regression in subjects relative to treatment with only the KRas G12D inhibitor. In one embodiment, the therapeutically effective amount of the combination of a PI3Ka inhibitor or a pharmaceutically acceptable salt thereof (for instance BYL719), and the KRas G12D inhibitor compound MRIX1133 or MRTX1133 analog or a pharmaceutically acceptable salt thereof, results in increased tumor growth inhibition in subjects relative to treatment with only the KRas G12D inhibitor. In one embodiment, the therapeutically effective amount of the combination of a PI3Ka inhibitor or a pharmaceutically acceptable salt thereof (for instance BYL719), and the KRas G12D inhibitor compound MRTX1133 or MRTX1133 analog or a pharmaceutically acceptable salt thereof, results in an improvement in the duration of stable disease in subjects compared to treatment with only the KRas G12D inhibitor. The amount of each compound in the combination may be the same or different than the therapeutically effective amount of each compound when administered alone as a monotherapy. Such amounts may be administered as a single dosage or may be administered according to a regimen, whereby it is effective.

As used herein, “treatment” means any manner in which the symptoms or pathology of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a KRas inhibitor or a pharmaceutically acceptable salt thereof, or the dose of irinotecan, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### KRas G12D Inhibitor Compounds

In one aspect of the invention, provided herein are methods of treating cancer in a subject in need thereof, comprising administering to the subject a therapeutically effective amount of a combination of irinotecan or an irinotecan analog or a pharmaceutically acceptable salt thereof, and the KRas G12D inhibitor compound MRTX1133 or a pharmaceutically acceptable salt or a pharmaceutical composition thereof.

In one embodiment, the KRas G12D inhibitor is:

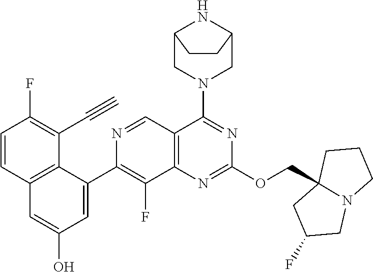 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

(also referred to as MRTX1133, and Example 252 in WO2021/041671) or a pharmaceutically acceptable salt thereof.

The KRas G12D inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12D inhibitor compound MRTX1133 used in the methods include salts of the above compounds, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the KRas G12D inhibitors disclosed herein are known. For example, WO2021/041671 describes general reaction schemes for preparing compounds including MRTX1133 and MRTX1133 analogs, and also provides detailed synthetic routes for the preparation of these compounds.

### PI3Ka Inhibitors

In one embodiment, the PI3Ka inhibitor or a pharmaceutically acceptable salt thereof of the present invention is BYL719 (alpelisib):

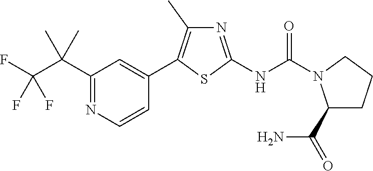 [SMILES: Cc1nc(NC(=O)N2CCC[C@H]2C(N)=O)sc1-c1ccnc(C(C)(C)C(F)(F)F)c1]

(2S)-N1-[4-Methyl-5-[2-(2,2,2-trifluoro-1,1-dimethylethyl)-4-pyridinyl]-2-thiazolyl]-1,2-pyrrolidinedicarboxamide

In one embodiment, the PI3Ka inhibitor or a pharmaceutically acceptable salt thereof of the present invention is selected from: BYL719, inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine); or a pharmaceutically acceptable salt thereof.

In another embodiment, the PI3Ka inhibitor or a pharmaceutically acceptable salt thereof of the present invention is selected from: alpelisib, GDC-0077, YM-201636, serabelisib, PIK-75 hydrochloride, GDC-0326, HS-173, A66, PF-4989216, pilaralisib analogue, PI-828, brevianamide F, PI3Kα-IN-4, BEBT-908, WYE-687, PF-06843195, CYH33, PI3Ky inhibitor 4, KU-0060648, WYE-687 dihydrochloride, PIK-75, PI3Kα/mTOR-IN-1, LY294002, AS-041164, idelalisib, buparlisib, dactolisib, pictilisib, eganelisib, copanlisib, duvelisib, fimepinostat, omipalisib, PI-103, taselisib, PF-04691502, ZSTK474, AZD 6482, samotolisib, dactolisib tosylate, AZD8186, AS-605240, copanlisib dihydrochloride, PKI-402, apitolisib, Vps34-PIK-III, gedatolisib, PIK-93, CH5132799, bimiralisib, GSK1059615, CNX-1351, BGT226 maleate, VS-5584, sonolisib, voxtalisib, PI4KIIIbeta-IN-9, leniolisib, nemiralisib, SF2523, AZD-8835, AMG 511, AZD3458, PIK-90, pictilisib dimethanesulfonate, AS-252424, AMG319, acalisib, pilaralisib, PI-103 Hydrochloride, SAR-260301, PI-3065, PQR530, hSMG-1 inhibitor 11j, GNE-477, PI3K/mTOR Inhibitor-2, buparlisib hydrochloride, MSC2360844, SRX3207, NSC781406, TG 100713, AS-604850, IPI-3063, PF-04979064, ETP-46321, GNE-493, PIK-294, (S)-PI3Kα-IN-4, PKI-179, PIK-293, CAL-130 hydrochloride, BGT226, PI3K-IN-6, PI3KS-IN-8, FD223, PARP/PI3K-IN-1, CHMFL-PI3KD-317, PI3K/HDAC-IN-1, PI3K-IN-2, PI3K/mTOR Inhibitor-1, PKI-179 hydrochloride, hSMG-1 inhibitor 11e, NVP-BAG956, PI3Kγ inhibitor 1, ON 146040, CAL-130, BAY1082439 and AZ2, or a pharmaceutically acceptable salt thereof.

The PI3Ka inhibitors of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation ofstereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In another embodiment, the PI3Ka inhibitors of the present invention include their salts, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing certain PI3Ka are well known and are disclosed, inter alia, in the patents recited herein.

### Pharmaceutical Compositions

PI3Ka inhibitors pharmaceutically acceptable salt thereof, and the KRas G12D compound MRTX1133 or MRTX1133 analogs, or pharmaceutically acceptable salts thereof, may be formulated into pharmaceutical compositions.

In another aspect, the invention provides pharmaceutical compositions comprising a PI3Ka inhibitor or a pharmaceutically acceptable salt thereof (for instance BYL719), and the KRas G12D compound MRTX1133 or MRTX1133 analogs, or pharmaceutically acceptable salts thereof, and one or more of a pharmaceutically acceptable carrier, excipient, or diluent that may be used in the methods disclosed herein, a PI3Ka inhibitor or a pharmaceutically acceptable salt thereof, and the KRas G12D compound MRTX1133 or MRTX1133 analogs, or pharmaceutically acceptable salts thereof, may be independently formulated by any method well known in the art and may be prepared for administration by any route, including, without limitation, parenteral, oral, sublingual, transdermal, topical, intranasal, intratracheal, intravenous or intrarectal. In certain embodiments, the two aforementioned components are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising a PI3Ka inhibitor or a pharmaceutically acceptable salt thereof, and the KRas G12D compound MRTX1133 or MRTX1133 analogs, or pharmaceutically acceptable salts thereof, may be used in the methods of use described herein.

### Co-Administration

The PI3Ka inhibitor or a pharmaceutically acceptable salt thereof (for instance BYL719), and the KRas G12D compound MRTX1133 or MRTX1133 analogs, or a pharmaceutically acceptable salt thereof, can be formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising a PI3Ka inhibitor or a pharmaceutically acceptable salt thereof (for instance BYL719), and the KRas G12D compound MRTX1133 or MRTX1133 analogs, or a pharmaceutically acceptable salt thereof, for use in the methods may be for simultaneous, separate or sequential use. In one embodiment, a PI3Ka inhibitor or a pharmaceutically acceptable salt thereof (for instance BYL719) is administered prior to administration of the KRas G12D compound MRTX1133 or MRTX1133 analog, or pharmaceutically acceptable salt thereof. In another embodiment, a PI3Ka inhibitor or a pharmaceutically acceptable salt thereof is administered after administration of the KRas G12D compound MRTX1133 or MRTX1133 analog or a pharmaceutically acceptable salt thereof. In another embodiment, a PI3Ka inhibitor or a pharmaceutically acceptable salt thereof is administered at about the same time as administration of the KRas G12D compound MRTX1133 or MRTX1133 analog or pharmaceutically acceptable salt thereof.

Separate administration of each inhibitor, at different times and by different routes, in some cases would be advantageous. Thus, the components in the combination, i.e., a PI3Ka inhibitor or a pharmaceutically acceptable salt thereof (for instance BYL719), and the KRas G12D compound MRTX1133 or MRTX1133 analogs, or pharmaceutically acceptable salt thereof, need not be necessarily administered at essentially the same time or in any order.

Oncology drugs are typically administered at the maximum tolerated dose (“MTD”), which is the highest dose of drug that does not cause unacceptable side effects. In one embodiment, a P3Ka inhibitor or a pharmaceutically acceptable salt thereof (for instance BYL719), and the KRas G12D compound MRTX1133 or MRTX1133 analog, or a pharmaceutically acceptable salt thereof, are each dosed at their respective MTDs. In one embodiment, a PI3Ka inhibitor or a pharmaceutically acceptable salt thereof is dosed at its MTD, and the KRas G12D compound MRTX1133 or MRTX1133 analog, or a pharmaceutically acceptable salt thereof, is dosed in an amount less than its MTD. In one embodiment, a PI3Ka inhibitor or a pharmaceutically acceptable salt thereof is dosed at an amount less than its MTD and the KRas G12D compound MRTX1133 or MRTX1133 analog, or a pharmaceutically acceptable salt thereof is dosed at its MTD. In one embodiment, the both components are each dosed at less than their respective MTDs. The administration can be so timed that the peak pharmacokinetic effect of one compound coincides with the peak pharmacokinetic effect of the other.

In one embodiment, a single dose of KRas G12D inhibitor compound MRTX1133 or MRTX1133 analog, or a pharmaceutically acceptable salt thereof, is administered per day (i.e., in about 24 hour intervals) (i.e., QD). In another embodiment, two doses of KRas G12D inhibitor compound MRTX1133 or MRTX1133 analog, or a pharmaceutically acceptable salt thereof, are administered per day (i.e., BID). In another embodiment, three doses of KRas G12D inhibitor compound MRTX1133 or MRTX1133 analog or a pharmaceutically acceptable salt thereof, are administered per day (i.e., TID).

In one embodiment, the PI3Ka inhibitor or a pharmaceutically acceptable salt thereof (for instance BYL719), is administered QD. In another embodiment the PI3Ka inhibitor or a pharmaceutically acceptable salt thereof is administered BID. In another embodiment the PI3Ka inhibitor or a pharmaceutically acceptable salt thereof of the invention are administered TID.

In one embodiment, a single dose of a PI3Ka inhibitor or a pharmaceutically acceptable salt thereof (for instance BYL719), and KRas G12D inhibitor compound MRTX1133 or MRTX1133 analog or a pharmaceutically acceptable salt or a pharmaceutical composition thereof, are each administered once daily.

Examples of a PI3Ka inhibitor or a pharmaceutically acceptable salt thereof suitable for the provided compositions and methods include those mentioned herein, for example: BYL719, inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine); or a pharmaceutically acceptable salt thereof.

Examples of KRas G12D inhibitors suitable for the provided compositions and methods include those mentioned herein, for example: MRTX1133, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; and pharmaceutically acceptable salts thereof.

### Combination Therapies

In one aspect of the invention, provided herein are methods of treating cancer in a subject in need thereof, comprising administering to the subject a therapeutically effective amount of a combination of a PI3Ka inhibitor or a pharmaceutically acceptable salt thereof (for instance BYL719), and the KRas G12D inhibitor compound MRTX1133 or an MRTX1133 analog, or a pharmaceutically acceptable salt or a pharmaceutical composition thereof. In one embodiment, the cancer is a KRas G12D-associated cancer. In one embodiment, the KRas G12D-associated cancer is pancreatic, colon, endometrial, or non-small cell lung cancer.

In yet another aspect, the invention provides for methods for increasing the sensitivity of a cancer cell to a KRas G12D inhibitor, comprising contacting the cancer cell with an effective amount of a combination of a PI3Ka inhibitor or a pharmaceutically acceptable salt thereof (for instance BYL719), and the KRas G12D inhibitor compound MRTX1133 or an MRTX1133 analog, or a pharmaceutically acceptable salt thereof, wherein the PI3Ka inhibitor or salt thereof increases the sensitivity of the cancer cell to the KRas G12D inhibitor. In one embodiment, the contacting is in vitro. In one embodiment, the contacting is in vivo.

In one embodiment, the combination therapy comprises a combination of a compound having the formula:

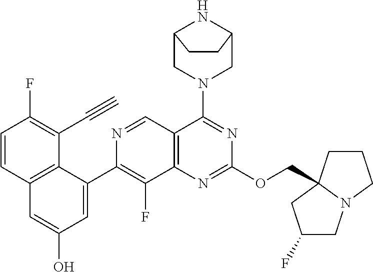 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

and a PI3Ka inhibitor or a pharmaceutically acceptable salt thereof (for instance BYL719).

In one embodiment, the combination therapy comprises a combination of a compound having the formula:

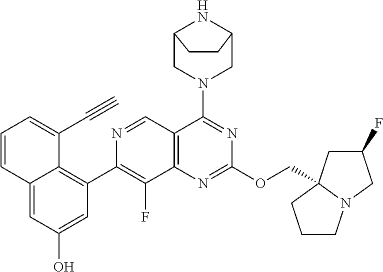 [SMILES: C#Cc1cccc2cc(O)cc(-c3ncc4c(N5CC6CCC(C5)N6)nc(OC[C@@]56CCCN5C[C@H](F)C6)nc4c3F)c12]

and a PI3Ka inhibitor or a pharmaceutically acceptable salt thereof.

In one embodiment, the combination therapy comprises a combination of a compound having the formula:

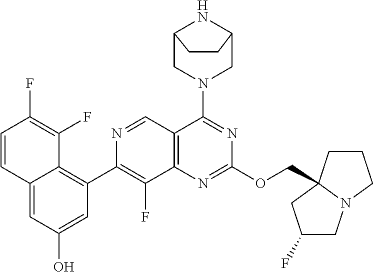 [SMILES: Oc1cc(-c2ncc3c(N4CC5CCC(C4)N5)nc(OC[C@@]45CCCN4C[C@H](F)C5)nc3c2F)c2c(F)c(F)ccc2c1]

and a PI3Ka inhibitor or a pharmaceutically acceptable salt thereof.

In one embodiment, the combination therapy comprises a combination of a compound having the formula:

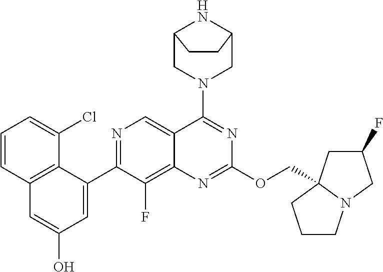 [SMILES: Oc1cc(-c2ncc3c(N4CC5CCC(C4)N5)nc(OC[C@@]45CCCN4C[C@H](F)C5)nc3c2F)c2c(Cl)cccc2c1]

and a PI3Ka inhibitor or a pharmaceutically acceptable salt thereof.

In one embodiment, the combination therapy comprises a combination of a compound having the formula:

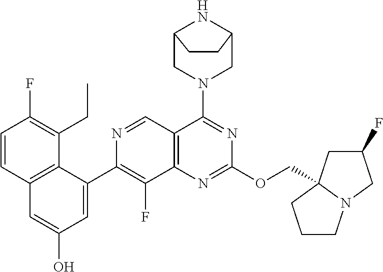 [SMILES: CCc1c(F)ccc2cc(O)cc(-c3ncc4c(N5CC6CCC(C5)N6)nc(OC[C@@]56CCCN5C[C@H](F)C6)nc4c3F)c12]

and a PI3Ka inhibitor or a pharmaceutically acceptable salt thereof.

In one embodiment, the combination therapy comprises a combination of a compound having the formula:

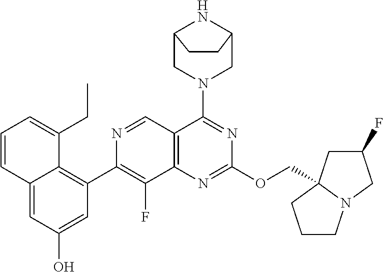 [SMILES: CCc1cccc2cc(O)cc(-c3ncc4c(N5CC6CCC(C5)N6)nc(OC[C@@]56CCCN5C[C@H](F)C6)nc4c3F)c12]

and a PI3Ka inhibitor or a pharmaceutically acceptable salt thereof.

In one embodiment, the combination therapy comprises a combination of a compound having the formula:

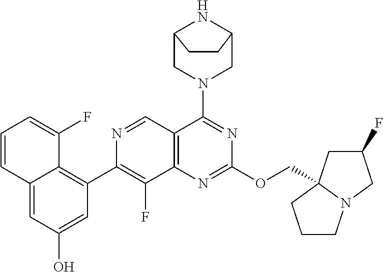 [SMILES: Oc1cc(-c2ncc3c(N4CC5CCC(C4)N5)nc(OC[C@@]45CCCN4C[C@H](F)C5)nc3c2F)c2c(F)cccc2c1]

and a PI3Ka inhibitor or a pharmaceutically acceptable salt thereof.

In one embodiment, the PI3Ka inhibitor or salt is BYL719, inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine); or a pharmaceutically acceptable salt thereof.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12D mutation, as well as, for example, introducing a combination provided herein into a sample containing a cellular or purified preparation containing KRas G12D mutation.

By negatively modulating the activity of KRas G12D, the methods described herein are designed to inhibit undesired cellular proliferation resulting from enhanced KRas G12D activity within the cell. The ability of a compound to inhibit KRas G12D may be monitored in vitro using well known methods, including those described in published international PCT application WO2021/041671. Likewise, the inhibitory activity of combination in cells may be monitored, for example, by measuring the inhibition of KRas G12D activity of the amount of phosphorylated ERK to assess the effectiveness of treatment and dosages may be adjusted accordingly by the attending medical practitioner.

The compositions and methods provided herein may be used for the treatment ofa KRas G12D-associated cancer in a subject in need thereof, comprising administering to said subject a therapeutically effective amount of a combination of a PI3Ka inhibitor or a pharmaceutically acceptable salt thereof (for instance BYL719), and the KRas G12D inhibitor compound MRTX1133 or MRTX1133 analog or a pharmaceutically acceptable salt thereof, wherein the PI3Ka inhibitor or salt increases the sensitivity the KRas G12D-associated cancer to the KRas G12D inhibitor. In one embodiment, the KRas G12C-associated cancer is pancreatic, colon, endometrial, or non-small cell lung cancer.

In one embodiment, the therapeutically effective amount of the combination of a PI3Ka inhibitor or a pharmaceutically acceptable salt thereof (for instance BYL719), and the KRas G12D inhibitor compound MRTX1133 or MRTX1133 analog or a pharmaceutically acceptable salt thereof, results in an increased duration of overall survival (“OS”) in subjects relative to treatment with only the KRas G12D inhibitor. In one embodiment, the therapeutically effective amount of a PI3Ka inhibitor or salt, and the KRas G12D inhibitor compound MRTX1133 or MRTX1133 analog or a pharmaceutically acceptable salt thereof, results in an increased duration of progression-free survival (“PFS”) in subjects relative to treatment with only the KRas G12D inhibitor. In one embodiment, the therapeutically effective amount of the combination of a PI3Ka inhibitor or salt, and the KRas G12D inhibitor compound MRTX1133 or MRTX1133 analog or a pharmaceutically acceptable salt thereof, results in increased tumor regression in subjects relative to treatment with only the KRas G12D inhibitor. In one embodiment, the therapeutically effective amount of the combination of a PI3Ka inhibitor or salt, and the KRas G12D inhibitor compound MRTX1133 or MRTX1133 analog or a pharmaceutically acceptable salt thereof, results in increased tumor growth inhibition in subjects relative to treatment with only the KRas G12D inhibitor. In one embodiment, the therapeutically effective amount of the combination of a PI3Ka inhibitor or salt, and the KRas G12D inhibitor compound MRTX1133 or MRTX1133 analog or a pharmaceutically acceptable salt thereof, results in an improvement in the duration of stable disease in subjects compared to treatment with only the KRas G12D inhibitor.

In another embodiment, a PI3Ka inhibitor or a pharmaceutically acceptable salt thereof (for instance BYL719) is administered in combination with the KRas G12D inhibitor compound MRTX1133 or MRTX1133 analog or a pharmaceutically acceptable salt thereof, once disease progression has been observed for KRas G12D monotherapy, in which the combination therapy results in enhanced clinical benefit for the patient by increasing OS, PFS, tumor regression, tumor growth inhibition or the duration of stable disease in the patient. In one embodiment, the KRas G12D inhibitor is a compound selected from MRX-′1133 and MRTX1133 analogs such as 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; and pharmaceutically acceptable salts thereof.

In one embodiment, the therapeutic combination comprises therapeutically effective amounts of MRTX1133 or a pharmaceutically acceptable salt thereof and BYL719 or a pharmaceutically acceptable salt thereof.

In one embodiment, the therapeutic combination comprises therapeutically effective amounts of MRTX1133 or a pharmaceutically acceptable salt thereof, and inavolisib, or a pharmaceutically acceptable salt thereof.

In one embodiment, the therapeutic combination comprises therapeutically effective amounts of MRTX1133 or a pharmaceutically acceptable salt thereof, and GDC-0326, or a pharmaceutically acceptable salt thereof.

In one embodiment, the therapeutic combination comprises therapeutically effective amounts of MRTX1133 or a pharmaceutically acceptable salt thereof, and GSK1059615, or a pharmaceutically acceptable salt thereof.

In one embodiment, the therapeutic combination comprises therapeutically effective amounts of MRTX1133 or a pharmaceutically acceptable salt thereof, and dactolisib, or a pharmaceutically acceptable salt thereof.

In one embodiment, the therapeutic combination comprises therapeutically effective amounts of MRTX1133 or a pharmaceutically acceptable salt thereof, and pictilisib, or a pharmaceutically acceptable salt thereof.

The compositions and methods provided herein may be used for the treatment of a wide variety of cancers including tumors such as pancreatic, colon, endometrial, and non-small cell lung cancer. The compositions and methods provided herein may also be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of a PI3Ka inhibitor or a pharmaceutically acceptable salt thereof (for instance BYL719), and the KRas G12D inhibitor compound MRTX1133 or MRTX1133 analog or a pharmaceutically acceptable salt thereof, wherein the PI3Ka inhibitor or salt increases the sensitivity of the KRas G12D-associated cancer to the KRas G12D inhibitor. In one embodiment, the KRas G12D inhibitor is a compound selected from MRX-1133 and MRTX1133 analogs such as 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; and pharmaceutically acceptable salts thereof.

In one embodiment, PI3Ka inhibitor or salt is BYL719, inavolisib (GDC-0077, (2S)-2-[[2-[(4S)-4-(difluoromethyl)-2-oxo-1,3-oxazolidin-3-yl]-5,6-dihydroimidazo[1,2-d][1,4]benzoxazepin-9-yl]amino]propanamide), GDC-0326 ((S)-2-((2-(1-isopropyl-1H-1,2,4-triazol-5-yl)-5,6-dihydrobenzo[f]imidazo[1,2-d][1,4]oxazepin-9-yl)oxy)propenamide), GSK1059615 (5-[[4-(4-pyridinyl)-6-quinolinyl]methylene]-2,4-thiazolidenedione), dactolisib (BEZ235, 2-methyl-2-[4-(3-methyl-2-oxo-8-quinolin-3-ylimidazo[4,5-c]quinolin-1-yl)phenyl]propanenitrile), and pictilisib (GDC-0941, 2-(1H-indazol-4-yl)-6-(4-methanesulfonyl-piperazin-1-ylmethyl)-4-morpholin-4-yl-thieno[3,2-d]pyrimidine); or a pharmaceutically acceptable salt thereof, is employed.

In a further embodiment, the therapeutic combination comprises therapeutically effective amounts of MRTX1133.

In a further embodiment, the therapeutic combination comprises therapeutically effective amounts of 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; or 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; and pharmaceutically acceptable salts thereof.

In one embodiment, the KRas G12D MRTX1133 or a pharmaceutically acceptable salt thereof, is administered as a parenteral, oral, sublingual, transdermal, topical, intranasal, intratracheal, intravenous or intrarectal formulation during a period of time. In one embodiment, the dose of MRTX1133 administered comprises on or more of: about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg and about 2000 mg. In one embodiment, MRTX1133 is administered once a day (QD) on a daily basis during a period of time. In one embodiment MRTX1133 is administered twice a day (BID) on a daily basis during a period of time.

In one embodiment, a PI3Ka inhibitor or a pharmaceutically acceptable salt thereof (for instance BYL719), is orally or intravenously administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg), in the case of intravenously over a period of time.

In one embodiment, 300 mg of the PI3Ka inhibitor BYL719 is orally administered daily.

In one embodiment, 250 mg of the PI3Ka inhibitor BYL719 is orally administered daily.

In one embodiment, 200 mg of the PI3Ka inhibitor BYL719 is orally administered daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20/c to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 36%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 500% c to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to, and/or provides, a kit comprising a PI3Ka inhibitor or pharmaceutically acceptable salt thereof, and the KRas G121) inhibitor compound MRTX1133 or MRTX1133 analog or a pharmaceutically acceptable salt thereof, for use in treating a cancer.

In a related aspect, the invention provides a kit containing a dose of a PI3Ka inhibitor or a pharmaceutically acceptable salt thereof, and dose of the KRas G12D inhibitor compound MRTX1133 or MRTX1133 analog or a pharmaceutically acceptable salt thereof, in an amount effective to inhibit proliferation of cancer cells, particularly KRas G12D-expressing cancer cells, in a subject. The kit in some cases includes an insert with instructions for administration of theses agents, where the insert may provide a user with one set of instructions for using these agents in combination.

The following Examples are intended to illustrate further certain embodiments of the invention and are not intended to limit the scope of the invention.

### Example A

### In Vivo Models for Examination of KRas G12D Inhibitor Plus PI3Ka Inhibitor Combinations

Immunocompromised nude/nude mice are inoculated in the right hind flank with cells harboring a KRas G12D mutation. When tumor volumes reach between 200-400 mm³ in size, the mice are divided into four to five groups of 5 mice each. The first group is administered vehicle only. The second group is administered a twice daily single agent dose of the KRas G12D inhibitor at a concentration that yields a maximal biological effect or a less than maximal biological effect, depending on the cell line and the single agent activity, that does not result in complete tumor regression. The second group, depending on cell line, may be administered a twice daily for 2 sequential days followed by 5 days off, the KRas G12D inhibitor at a concentration that yields a maximal biological effect or a less than maximal biological effect, depending on the cell line and single agent activity, that does not result in complete tumor regression. The third group is administered a single agent dose of BYL719 (Alpelisib) at a concentration that yields a maximal biological effect or a less than maximal biological effect, depending on the cell line and the single agent activity, that also does not result in complete tumor regression. The fourth group is administered the single agent dose of the KRas G12D inhibitor using the twice daily for 2 sequential days followed by 5 days off schedule in combination with the single agent dose of Irinotecan. The treatment period varies from cell line to cell line but typically is between 15-22-days. Tumor volumes are measured using a caliper every two-three days and tumor volumes are calculated by the formula: 0.5×(Length×Width)². A greater degree of tumor growth inhibition for the combination in this model demonstrates that the combination therapy is likely to have a clinically meaningful benefit to treated subjects relative to treatment with only a KRas G12D inhibitor.

20 to 25 nude/nude mice per study were inoculated in the right hind limb with 5×106 LS180 cells, AsPC-1 cells, GP2D cells, or Panc 02.03 cells. When tumor volumes reached ˜200 mm3-400 mm3 (study day 0) 5 mice in each of the groups were administered i.p. vehicle only (10% captisol in 50 mM citrate buffer pH 5.0), 30 mg/kg of Kras G12D inhibitor MRTX1133 (10% captisol in 50 mM citrate buffer, pH 5.0) either on the twice daily schedule or the twice daily for 2 consecutive days followed by 5 days off schedule, 15 mg/kg once daily of the BYL719 (0.5% methylcellulos) PI3Ka inhibitor, or 30 mg/kg of Kras G12D inhibitor on either schedule and BYL719. Tumor volumes, measured at pre-specified days, for the five mice per group were averaged and are reported for LS180, AsPC-1, GP2D, and Panc 02.03 in Tables 1, 2, 3, and 4, respectively.

### Example B

KRas G12D Inhibitor MRTX1133 in Combination with BYL719 (LS180 Colon Cancer Cell Line)

25 nude/nude mice were inoculated with LS180 cells in the hind right flank. When the tumors reached ˜250 mm³ five treatment groups were established with 5 mice per group. The results of this study are provided in Table 1:

TABLE 1
Average Tumor Volumes (mm³) of nude/nude Tumor Bearing
Mice Treated with Single Agents and in Combination

| | | | MRTX1133 | | |
|---|---|---|---|---|---|
| | Vehicle | MRTX1133 | 30 mg/kg | BLY719 | BLY719 + |
| Study | BID | 30 mg/kg | BID | 15 mg/kg | MRTX1133 |
| Day | Daily | BID Daily | 2×/week | QD | 2×/week |
| 1 | 268.842 | 268.94 | 272.338 | 273.184 | 275.41 |
| 4 | 601.218 | 418.808 | 570.528 | 501.45 | 347.854 |
| 8 | 1089.326 | 593.71 | 1134.668 | 799.338 | 436.068 |
| 11 | 1738.074 | 808.808 | 1439.99 | 1016.09 | 541.39 |
| 15 | 1965.054 | 1197.106 | 1899.832 | 1225.468 | 720.182 |

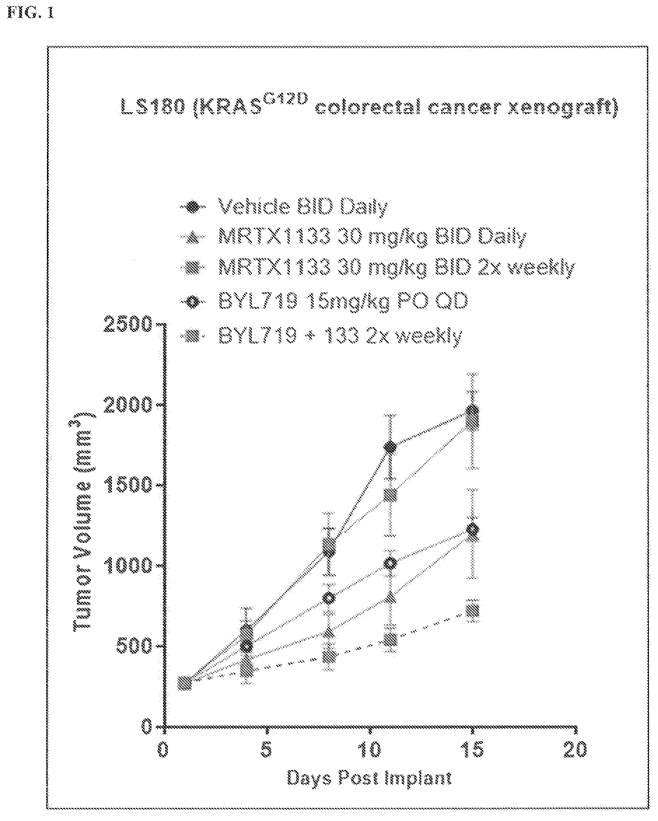

As shown in Table 1, the administration of MRTX1133 at 30 mg/kg BID (twice per day) as a single agent exhibited 45% tumor growth inhibition at Day 15 (daily administration) and 4% tumor growth inhibition at Day 15 (twice per week administration). The administration of PI3K inhibitor BYL719 at 15 mg/kg once daily as a single agent exhibited 44% tumor growth inhibition at Day 15. The combination of PI3K inhibitor BYL719 and MRTX1133 administered twice per week resulted in 73% growth inhibition at Day 15. See FIG. 1.

### Example C

KRas G12D Inhibitor MRTX1133 in Combination with BLY719 (AsPC-1 Pancreatic Cancer Cell Line)

30 nude/nude mice were inoculated with AsPC-1 cells in the hind right flank. When the tumors reached ˜300 mm³ six treatment groups were established with 5 mice per group. The results of this study are provided in Table 2:

TABLE 2
Average Tumor Volumes (mm³) of nude/nude Tumor Bearing
Mice Treated with Single Agents and in Combination

| | | MRTX1133 | MRTX1133 | BYL719 | BYL719 + | BYL719 + |
|---|---|---|---|---|---|---|
| Study | Vehicle | 30 mg/kg | 30 mg/kg | 15 mg/kg | MRTX1133 | MRTX1133 |
| Day | BID Daily | BID Daily | BID 2×/week | QD | BID Daily | 2×/week |
| 1 | 325.54208 | 323.454 | 325.894 | 331.29 | 332.47 | 333.582 |
| 6 | 460.79656 | 356.398 | 394.182 | 429.172 | 311.298 | 356.498 |
| 9 | 545.79776 | 374.684 | 472.586 | 473.138 | 277.396 | 436.136 |
| 13 | 618.26576 | 316.51 | 496.598 | 531.394 | 224.732 | 497.8 |
| 16 | 758.40264 | 291.71 | 554.052 | 580.446 | 175.634 | 520.042 |
| 20 | 857.15704 | 269.246 | 586.5625 | 688.564 | 116.562 | 511.334 |
| 23 | 949.84696 | 213.334 | 640.49 | 742.98 | 109.852 | 560.656 |
| 27 | 1047.63768 | 195.354 | 679.96 | 918.424 | 96.592 | 567.53 |
| 30 | 1109.212 | 216.072 | 795.11 | 1282.95667 | 133.766 | 605.776 |
| 34 | 1182.71952 | 266.324 | 862.74 | 1622.18667 | 180.834 | 669.406 |

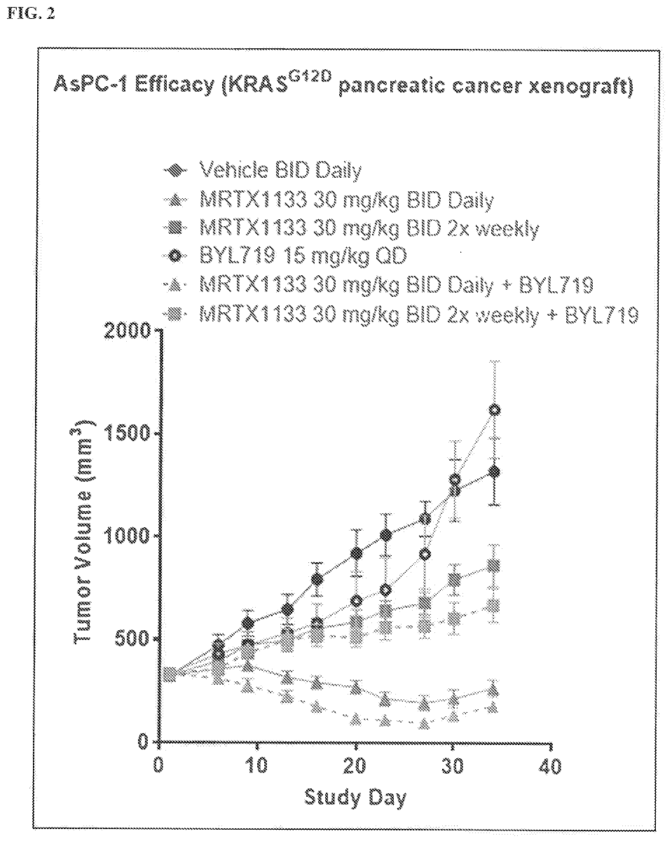

As shown in Table 2, the administration of MRTX1133 as a single agent (30 mg/kg BID daily) exhibited −9% tumor regression at day 34. The administration of PI3K inhibitor BYL719 at 15 mg/kg once daily as a single agent exhibited 0% tumor growth inhibition at Day 34. The combination of PI3K inhibitor BYL719 and MRTX1133 administered BID daily resulted in −46% tumor regression at Day 34. The administration of MRTX1133 as a single agent (30 mg/kg BID twice weekly) exhibited 43% tumor growth inhibition at day 34. The combination of MRTX1133 (30 mg/kg BID twice weekly) and BYL719 resulted in a 65% tumor growth inhibition at day 34. See FIG. 2.

### Example D

KRas G12D Inhibitor MRTX1133 in Combination with BYL719 (GP2D Colorectal Cancer Cell Line)

20 nude/nude mice were inoculated with GP2D cells in the hind right flank. When the tumors reached ˜300 mm³ four treatment groups were established with 5 mice per group. The results of this study are provided in Table 3:

TABLE 3
Average Tumor Volumes (mm³) of nude/nude Tumor
Bearing Mice Treated with Single Agents and in Combination

| | Vehicle | MRTX1133 | BYL719 | BYL719 + |
|---|---|---|---|---|
| Study | BID | 30 mg/kg | 15 mg/kg | MRTX1133 BID |
| Day | Daily | BID Daily | QD | Daily |
| 1 | 299.576 | 300.778 | 307.604 | 318.162 |
| 7 | 356.556 | 263.132 | 369.474 | 243.966 |
| 10 | 402.696 | 237.628 | 395.68 | 226.618 |
| 14 | 511.574 | 221.728 | 467.526 | 183.378 |
| 17 | 627.104 | 221.788 | 546.962 | 133.908 |
| 21 | 798.57 | 242.56 | 681.056 | 151.062 |
| 26 | 1179.594 | 254.57 | 901.144 | 149.842 |
| 28 | 1354.81 | 307.03 | 1053.732 | 154.004 |
| 31 | 1589.53 | 333.20 | 1102.87 | 147.39 |
| 35 | 1912.86 | 358.75 | 1202.89 | 171.49 |

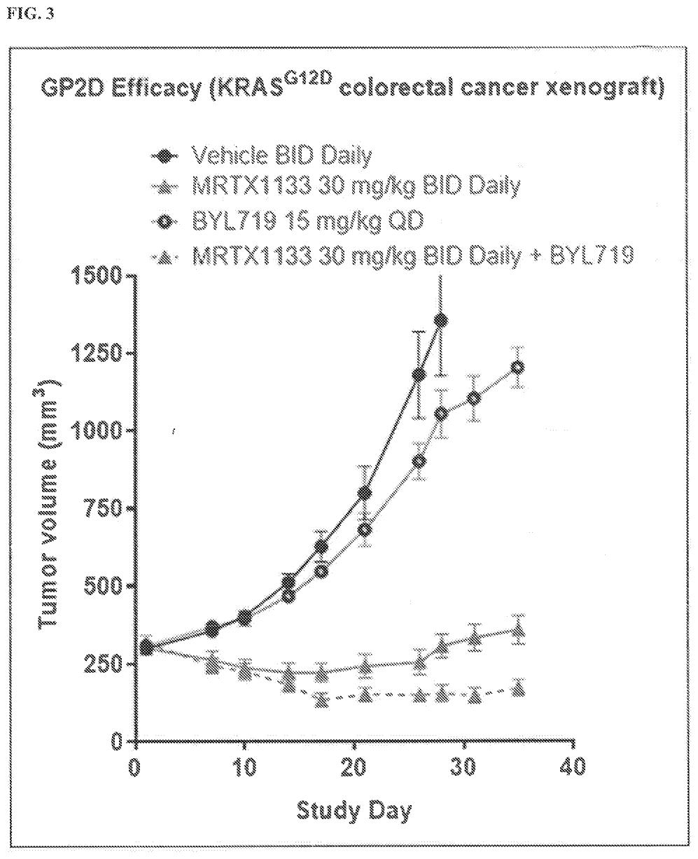

As shown in Table 3, the administration of MRTX1133 as a single agent exhibited 96% tumor growth inhibition at day 35. The combination of MRTX1133 and BYL719 resulted in a −46% tumor regression at day 35. See FIG. 3.

### Example E

KRas G12D Inhibitor MRTX1133 in Combination with BYL719 (PANC0203 Pancreatic Cancer Cell Line)

20 nude/nude mice were inoculated with Panc 02.03 cells in the hind right flank. When the tumors reached ˜300 mm³ four treatment groups were established with 5 mice per group. The results of this study are provided in Table 4:

TABLE 4
Average Tumor Volumes (mm³) of nude/nude Tumor
Bearing Mice Treated with Single Agents and in Combination

| | Vehicle | MRTX1133 | BYL719 | BYL719 + |
|---|---|---|---|---|
| Study | BID | 30 mg/kg BID | 15 mg/kg | MRTX1133 |
| Day | Daily | 2×/week | QD | 2×/week |
| 1 | 328.896 | 329.18 | 330.188 | 332.236 |
| 4 | 446.116 | 265.246 | 341.414 | 245.512 |
| 8 | 544.424 | 386.354 | 367.35 | 223.012 |
| 11 | 647.874 | 392.042 | 487.664 | 222.548 |
| 15 | 786.634 | 480.434 | 572.82 | 282.214 |
| 18 | 927.746 | 496.496 | 680.306 | 305.212 |
| 22 | 1113.464 | 549.618 | 774.992 | 342.742 |

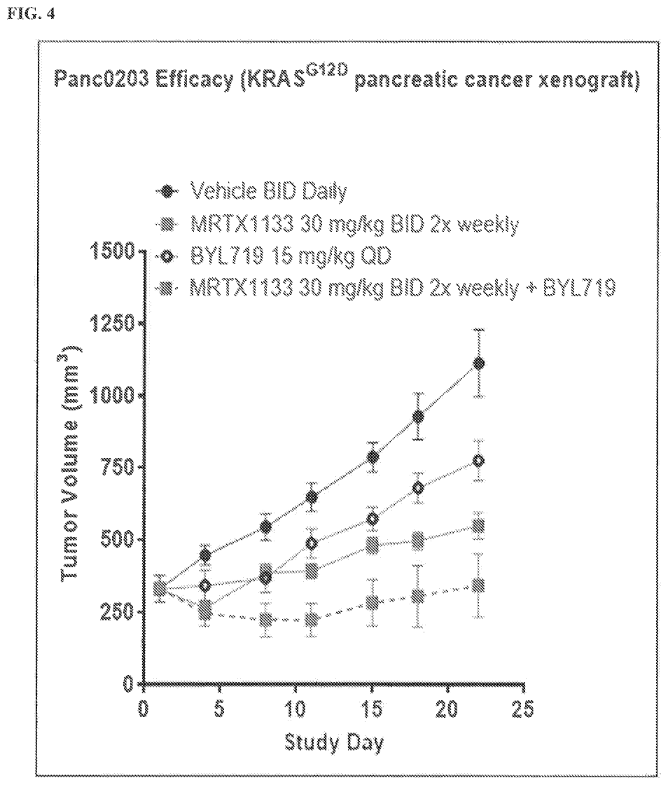

As shown in Table 4, the administration of MRTX1133 as a single agent exhibited 72% tumor growth inhibition at day 22. The combination of MRTX1133 and BYL719 resulted in 98% tumor growth inhibition at day 22. See FIG. 4.

### Example F

In Vitro Data Demonstrating Synergy of MRTX1133 in Combination with BYL719

A panel of KRAS G12D mutant cell lines was used to identify synergistic combinations with KRAS G12D inhibitor, MRTX1133. The cells were grown in a monolayer in a 2D, with drug treatment for 72 hours. The dilutions used for the KRAS G12D inhibitor MRTX1133 and the combination partner varied for each compound but were in the range of 3-to 6-fold/serial dilution. Each single agent and the associated combinations of the dose matrix was added and the plates were incubated for 72 hours at 370C in 5% CO₂ atmosphere. End-point Cell-Titer-Glow (CTG) reads were generated to determine viability of each single agent and the combination.

A custom R-script was created, integrating open source Bioconductor packages, to batch process metadata files containing experimental parameters and raw data files. Various numerical and graphical outputs were generated to summarize the data. Single agent parameters were generated using GRmetrics (Hafner M et al.) while the synergyfinder package was used to determine whether the two test compounds demonstrate synergy using four independent mathematical reference models (Loewe additivity, Bliss independence, Highest Single Agent and ZIP) (He L et al.). The output of the data from each mathematical model is the assignment of a relative synergy score. The data reported in the table are the aggregate sum of the Loewe additivity, Bliss independence, Highest Single Agent and ZIP scores (“Composite Synergy Score”). Composite Synergy Score 22-80=synergy. Composite Synergy Score 11-21=additive. Composite Synergy Score <0-10=no benefit.

The results of this study are provided in Table 5:

TABLE 5
Synergy scores for combination
of MRTX1133 and BLY719
Combination: KRas G12D
Inhibitor MRTX1133
and PI3Ka Inhibitor BLY719

| Cell Line: | Synergy Score: |
|---|---|
| SNU61 | 9 |
| LS180 | 18 |
| Panc0504 | 17 |
| Panc0203 | 77 |
| SNU407 | 23 |
| LS513 | 14 |
| A427 | 51 |
| HPAC | 44 |
| AGS | 64 |
| SNU1197 | 30 |
| SNU1033 | 26 |
| SNU410 | 8 |
| Hec1B | 21 |
| SU8684 | 12 |
| SNUC2B | −5 |
| Panc0813 | 28 |
| SUIT2 | 6 |
| HPAFII | 27 |
| Panc0403 | 34 |
| Panc1005 | 34 |
| HCC1588 | 2 |
| GP2D | 49 |
| ASPC1 | 16 |
| SW1990 | 34 |
| KP4 | 41 |
| Panc1 | −4 |

These results demonstrate that the combination is synergistic for the majority of cell lines tested, and additive for the majority of the remaining cell lines tested.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of treating cancer in a subject in need thereof, comprising administering to the subject a therapeutically effective amount of a combination a KRas G12D inhibitor or a pharmaceutically acceptable salt thereof, and a PI3Ka inhibitor or a pharmaceutically acceptable salt thereof.

2. The method of claim 1, wherein the KRas G12D inhibitor or salt is selected from: MRTX1133, 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-fluoronaphthalen-2-ol; and pharmaceutically acceptable salts thereof.

3. The method of claim 1, wherein the KRas G12D inhibitor is MRTX1133: 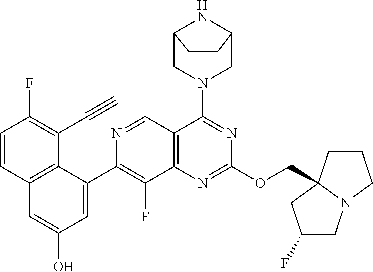 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, and the PI3Ka inhibitor is BYL719: 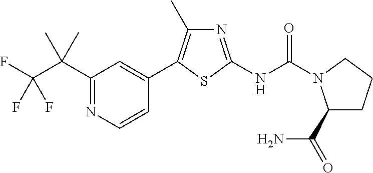 [SMILES: Cc1nc(NC(=O)N2CCC[C@H]2C(N)=O)sc1-c1ccnc(C(C)(C)C(F)(F)F)c1]
or a pharmaceutically acceptable salt thereof.

4. The method of according to claim 1, wherein the KRas G12D inhibitor or salt, and the PI3Ka inhibitor or salt, are administered on the same day.

5. The method of according to claim 1, wherein the KRas G12D inhibitor or salt, and the PI3Ka inhibitor or salt, are administered on different days.

6. The method according to claim 1, wherein the KRas G12D inhibitor or salt is administered at a maximum tolerated dose.

7. The method according to claim 1, wherein the PI3Ka inhibitor or salt is administered at a maximum tolerated dose.

8. The method according to claim 1, wherein the KRas G12D inhibitor or salt, and the PI3Ka inhibitor or salt, are each administered at a maximum tolerated dose.

9. The method according to claim 1, wherein the KRas G12D inhibitor or salt is administered at below maximum tolerated dose.

10. The method according to claim 1, wherein the PI3Ka inhibitor or salt is administered at below maximum tolerated dose.

11. The method according to claim 1, wherein the KRas G12D inhibitor or salt, and the PI3Ka inhibitor or salt, are each administered at below maximum tolerated dose.

12. The method according to claim 1, wherein the therapeutically effective amount of the combination of the KRas G12D inhibitor or salt and the PI3Ka inhibitor or salt results in an increased duration of overall survival, an increased duration of progression free survival, an increase in tumor growth regression, an increase in tumor growth inhibition or an increased duration of stable disease in the subjects relative to treatment with only the KRas G12D inhibitor or salt.

13. The method according to claim 1, wherein the therapeutically effective amount of the combination of the KRas G12D inhibitor or salt, and the PI3Ka inhibitor or salt, results in an increased duration of overall survival, an increased duration of progression free survival, an increase in tumor growth regression, an increase in tumor growth inhibition or an increased duration of stable disease in the subjects relative to treatment with only the cytotoxic compound.

14. A pharmaceutical composition comprising a therapeutically effective amount of a combination of a KRas G12D inhibitor or pharmaceutically acceptable salt thereof, and a PI3Ka inhibitor or salt, and a pharmaceutically acceptable excipient.

15. The composition of claim 14, comprising MRTX1133: 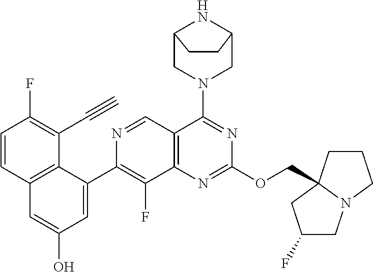 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, and
BYL719: 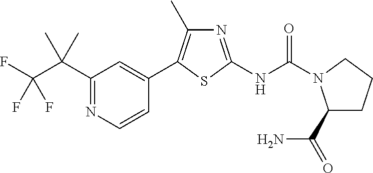 [SMILES: Cc1nc(NC(=O)N2CCC[C@H]2C(N)=O)sc1-c1ccnc(C(C)(C)C(F)(F)F)c1]
or a pharmaceutically acceptable salt thereof, and a pharmaceutically acceptable excipient.

16. A method for inhibiting KRas G12D activity in a cell, comprising contacting the cell in which inhibition of KRas G12D activity is desired with an effective amount of a combination a KRas G12D inhibitor or a pharmaceutically acceptable salt thereof, and a PI3Ka inhibitor or a pharmaceutically acceptable salt thereof.

17. The method of claim 16, wherein the KRas G12D inhibitor is MRTX1133: 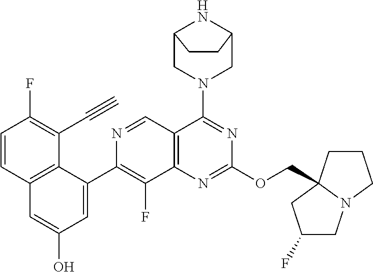 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
and pharmaceutically acceptable salts thereof, and the PI3Ka inhibitor is BYL719: 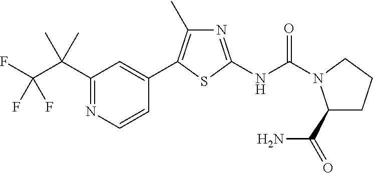 [SMILES: Cc1nc(NC(=O)N2CCC[C@H]2C(N)=O)sc1-c1ccnc(C(C)(C)C(F)(F)F)c1]
or a pharmaceutically acceptable salt thereof

18. The method according to claim 1, wherein the PI3Ka inhibitor increases the sensitivity of cancer cells to the KRas G12D inhibitor.

19. A method for increasing the sensitivity of a cancer cell to the KRas G12D inhibitor comprising administering to a subject undergoing KRas G12DC treatment with an effective amount of a combination the KRas G12C inhibitor MRTX1133: 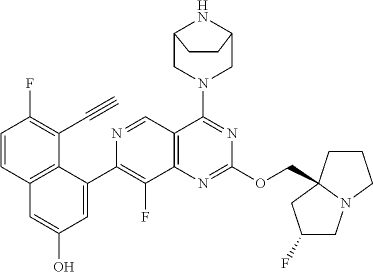 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, and BYL719: 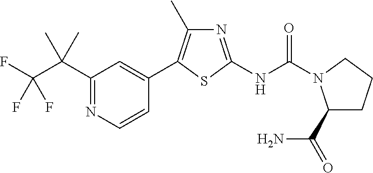 [SMILES: Cc1nc(NC(=O)N2CCC[C@H]2C(N)=O)sc1-c1ccnc(C(C)(C)C(F)(F)F)c1]
or a pharmaceutically acceptable salt thereof, wherein the BYL719 increases the sensitivity of the cancer cell to the KRas G12D inhibitor.

20. The method according to claim 1, wherein the therapeutically effective amount of the KRas G12D inhibitor in the combination is between about 0.01 to 100 mg/kg per day.

21. The method of claim 20, wherein the therapeutically effective amount of the KRas G12D inhibitor in the combination is between about 0.1 to 50 mg/kg per day.

22. The method according to claim 1, wherein the therapeutically effective amount of PI3Ka inhibitor or salt in the combination is between about 0.01 to 100 mg/kg per day.

23. The method of claim 22, wherein the therapeutically effective amount of PI3Ka inhibitor or salt in the combination is between about 0.1 to 50 mg/kg per day.

24. The method according to claim 22, wherein the PI3Ka inhibitor or salt os BYL719.

25. The method according to claim 1, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

26. The method of claim 25, wherein the cancer wherein the cancer is a KRas G12D-associated cancer.

27. The method of claim 26, wherein the cancer is pancreatic, colon, endometrial, or non-small cell lung cancer.

28. A kit comprising the pharmaceutical composition of claim 14 for treating KRas G12D cancer in a subject.

29. The kit according to claim 28, further comprising an insert with instructions for administration of the pharmaceutical composition.
